(19) United States
(12) Patent Application Publication
(10) Pub. No.: US 2007/0191287 A1
(43) Pub. Date: Aug. 16, 2007

(54) D-ribose for improving depression-like symptoms

(76) Inventors: Michio Yamamura, Osaka-shi (JP); Koukichi Hayashida, Osaka-shi (JP); Toshito Tsuchida, Osaka-shi (JP)

Correspondence Address: BIRCH STEWART KOLASCH & BIRCH, PO BOX 747, FALLS CHURCH, VA, 22040-0747, US

(21) Appl. No.: 10/590,986

(22) PCT Filed: Mar. 17, 2005
(86) PCT No.: PCT/JP05/05452

§ 371 Date: Aug. 29, 2006

(30) Foreign Application Priority Data
Mar. 18, 2004 (JP) 2004-078521
Apr. 22, 2004 (JP) 2004-126176
Sep. 30, 2004 (JP) 2004-287677

(51) Int. Cl. A61K 31/70; A61K 31/205; A61K 31/198

(52) U.S. Cl. 514/23; 514/566; 514/554

(57) ABSTRACT

An agent for improving depression-like symptoms comprising D-ribose, which may improve an alleviate various symptoms such as hypobulia, general fatigue, sluggishness, enervation, deterioration in concentration, memory impairment, abnormal sensation/obtundation such as impaired sight, decline in thinking power, indefinite complaint, drop in operation efficiency, or feeling of malaise, etc.

### TECHNICAL FIELD

The present invention relates to an agent for improving depression-like symptoms.

### BACKGROUND ART

Social stress and psychologic stress are more and more increased in modern societies, and by undergoing such high stress, there is an increase in the number of people who complain fatigue or are suffered from hypobulia, general fatigue, sluggishness, enervation, deterioration in concentration, memory impairment, abnormal sensation/obtundation such as impaired sight, decline in thinking power, indefinite complaint, drop in operation efficiency, or feeling of malaise, etc. According to the results of the epidemiological investigation for 4, 000 persons of different sexes between of the age of 15 to 65, which was carried out by Health and Welfare Ministry, the survey research group for fatigue on 1999 in Japan, it has been revealed that about 60% of the subjects to be investigated complained fatigue/weariness, and about one out of every three complains chronic fatigue, which lasts for 6 months or more or frequently repeats (cf., Igaku-no-Ayumi, i.e., Development of medical science, 204 (5), p. 381-386 (2003)). Thus, chronic fatigue and chronic fatigue syndrome have become a serious social issue. These chronic fatigue, etc. have been considered to be caused by psychoneurotic factors such as stress, or by chronic infection or an indefinite infection factor, etc., and then they may be exacerbated by complexly intertwining with various related factors (cf., Igaku-no-Ayumi, 204 (5), p. 381-386 (2003)).

By the way, various pharmacological effects of D-ribose have been reported. For example, JP-A-2002-518321 discloses that D-ribose increases the energy level in a mammal by stimulating the synthesis of ATP. Further, it has been reported that D-ribose is effective for a patient of coronary heart disease (cf., Lancet, 340, p. 507-510 (1992)), or for a patient of epilepsy (cf., Biochimica et Biophisica Acta, 1453, p. 135-140 (1999)). However, it has not been known at the moment that D-ribose exhibits an effect of improving depression-like symptoms.

### DISCLOSURE OF INVENTION

An object of the present invention is to provide an excellent agent for improving depression-like symptoms.

The present inventors have intensively studied on an agent for improving depression-like symptoms, and they have surprisingly found that D-ribose exhibits an excellent and significant improving activity in the forced swimming test in mice and the reserpine-induced hypothermia competitive test, which are regarded as tests for predictions of possible clinical antidepressant activity, and have accomplished the present invention.

That is, the present invention comprises the following embodiments:

[1] An agent for improving depression-like symptoms, which comprises D-ribose.

[2] The agent for improving depression-like symptoms according to the above [1], wherein the depression-like symptoms are hypobulia, general fatigue, sluggishness, enervation, deterioration in concentration, memory impairment, abnormal sensation/obtundation such as impaired sight, decline in thinking power, indefinite complaint, drop in operation efficiency, or feeling of malaise.

[3] The agent for improving depression-like symptoms according to the above [1] or [2], wherein the depression-like symptoms are depression-like symptoms accompanied by mental overstrain or mental disorder.

[4] The agent for improving depression-like symptoms according to any one of the above [1] to [3], which comprises D-ribose in an amount of 10 mg to 100 g per day for an adult.

[5] The agent for improving depression-like symptoms according to any one of the above [1] to [4], which further comprises at least one of a magnesium salt, an amino acid and carnitine.

[6] The agent for improving depression-like symptoms according to any one of the above [1] to [4], which further comprises potassium magnesium aspartate.

[7] A composition, which comprises D-ribose together with at least one of a magnesium salt, an amino acid and carnitine.

[8] A food or drink for improving depression-like symptoms, which comprises D-ribose.

[9] An agent for improving mental fatigue, which comprises D-ribose.

### BRIEF DESCRIPTION OF DRAWINGS

FIG. 1 shows the results of measuring the duration of immobility in the groups treated with D-ribose in the forced swimming test in mice.

* P<0.05; ** P<0.01 vs Control group (Dunnett's multiple comparison test)

FIG. 2 shows the results of measuring the duration of immobility in the groups treated with a combination of D-ribose and potassium magnesium aspartate in the forced swimming test in mice.

* P<0.05; ** P<0.01 vs Control group (Dunnett's multiple comparison test)

FIG. 3 shows the antagonistic effect of D-ribose on hypothermia in the reserpine-induced hypothermia competitive test.

* P<0.05; ** P<0.01 vs Control group (Bonferroni/Dunn's multiple comparison test)

FIG. 4 shows the antagonistic effect of a combination of D-ribose and potassium magnesium aspartate on hypothermia in the reserpine-induced hypothermia competitive test.

* P<0.05; ** P<0.01 vs Control group (Bonferroni/Dunn's multiple comparison test)

FIG. 5 shows the results of measuring the duration of swimming in the groups treated with D-ribose in the soaking fatigue test in rats.

*P<0.05; ** P<0.01 vs Control group (Bonferroni/Dunn's multiple comparison test)

FIG. 6 shows the results of measuring the duration of immobility in the groups treated with D-ribose and glucose in the forced swimming test in mice.

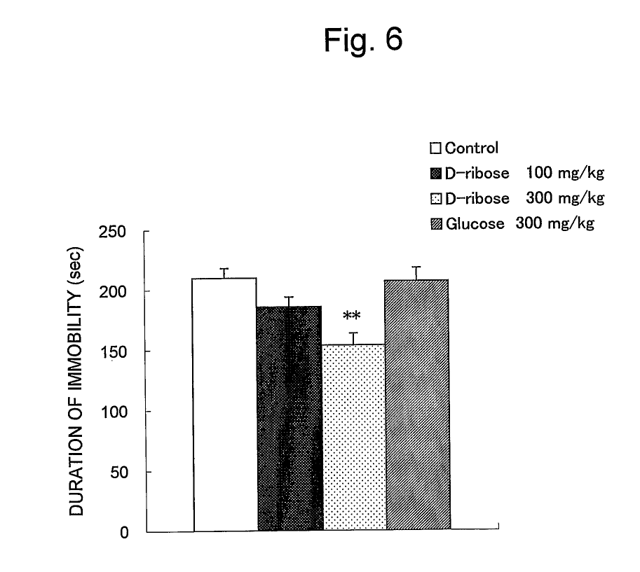

** P<0.01 vs Control group (Bonferroni/Dunn's multiple comparison test)

### BEST MODE FOR CARRYING OUT THE INVENTION

In the present invention, D-ribose includes derivatives of D-ribose and any other derivatives which may be converted into D-ribose in vivo.

The “depression-like symptoms” include hypobulia, general fatigue, sluggishness, enervation, deterioration in concentration, memory impairment, and abnormal sensation/obtundation such as impaired sight, decline in thinking power, indefinite complaint, drop in operation efficiency, or feeling of malaise, etc. Further, the “depression-like symptoms” include depression-like symptoms accompanied by mental overstrain or mental disorder, etc. The “depression-like symptoms accompanied by mental overstrain” include, for example, depression-like symptoms in persons who are suffered from chronic fatigue syndrome and chronic fatigue, etc. The “depression-like symptoms accompanied by mental disorder” include, for example, depression-like symptoms accompanied by endogenous mental disorder, depression-like symptoms accompanied by psychogenic mental disorder, or depression-like symptoms accompanied by exogenous mental disorder, etc. The “endogenous mental disorder” is, for example, depression, schizophrenia, etc. The “psychogenic mental disorder” is, for example, neurosis (anxiety neurosis, depressive neurosis), panic disorders, post-traumatic stress disorders, sleep disturbance, etc. The “exogenous mental disorder” is, for example, brain organic mental disorders, neurodegenerative disorders, etc., such as Alzheimer's disease, senile dementia, Parkinson's disease, Huntington's chorea, cerebrovascular disease aftereffects, head injury aftereffects, alcoholics, migraine, thyroid hypofunction, adrenal insufficiency, cancer such as brain cancer, fatal disease, etc.

D-ribose may improve depression-like symptoms, depression-like symptoms accompanied by mental overstrain or depression-like symptoms accompanied by mental disorder, etc. Further, by improving depression-like symptoms accompanied by mental overstrain, D-ribose may be expected to improve immune abnormality accompanied by chronic fatigue or chronic fatigue syndrome, for example, chronic infection such as cold or chlamydia mycoplasma, revitalization of latent infection virus such as human herpesvirus EB virus (Epstein-Barr virus), etc., which are caused by immune compromise in NK activity.

Magnesium salt includes, for example, magnesium salt of an amino acid such as aspartic acid, glutamic acid, valine, leucine, isoleucine, etc.; magnesium salt of an organic acid such as acetic acid, stearic acid, docosahexaenoic acid, citric acid, oxalic acid, tartaric acid, etc.; and a magnesium salt of an inorganic acid such as hydrochloric acid, sulfuric acid, phosphoric acid, etc. Preferable magnesium salt is, for example, a magnesium salt of aspartic acid or glutamic acid, phosphoric acid, etc., and more preferable magnesium salt is magnesium aspartate, potassium magnesium aspartate, trimagnesium phosphate, etc. A magnesium salt may enhance the improving effect of D-ribose on depression-like symptoms, and hence, even at a dose not enough to exhibit a sufficient effect when administered alone, D-ribose may improve depression-like symptoms by administering together with a magnesium salt.

Further, a magnesium salt-containing food or drink may be used instead of a magnesium salt, and such foods or drinks are exemplified below.

(1) Cereals: wheat, rice, buckwheat, corn, foxtail millet, oatmeal, millet, rye, etc.

(2) Potato and starch: Irish potato, sweet potato, etc.

(3) Nuts and seeds: almonds, hempseeds, cashew nuts, sesame seeds, pistachios, macadamia nuts, pine nuts, peanuts, etc.

(4) Beans: adzuki beans, kidney beans, peas, cowpeas, soybeans, etc.

(5) Fish and seafood: scad, dried sardine, young sardine, true sardine, bonito, salmon, arch shell, clam, turban shell, tokobushi abalone, scallop, surf clam, mysid, shrimp, crab, sea cucumber, etc.

(6) Animal, chicken and whale meats: beef, pork, chicken, mutton, etc.

(7) Milk products: cow milk, fermented milk, yogurt, ice cream, powdered skim milk, cheese, etc.

(8) Vegetables: barilla, dried gourd shavings, beefsteak plant, aroid, spinach, etc.

(9) Fungus: shiitake mushroom, etc.

(10) Algae: green layer, sweet layer, Arame (blackish brown seaweed), false Ceylon moss, tangle, agar-agar, edible brown algae, nitidum Wittrock, brown seaweed, etc.

(11) Beverages: tea, cocoa, tangle tea, ocean deep water, etc.

(12) Seasonings and spices: bittern, salt, soy sauce, mustard, curry, pepper, zanthoxyli fructus, ginger, red pepper, yeast, etc.

(13) Others: dolomite

When administered together with the above-mentioned foods and drinks, D-ribose may be taken in the form of a food or drink, which is prepared from the above-mentioned magnesium salt-containing foods or drinks, or an extract thereof, and D-ribose is used therein as a sweetening. These processed foods and drinks may be any ones as long as they are prepared from the above-mentioned magnesium salt-containing foods and drinks, and include, for example, various sweet stuffs such as pie, cracker, chips, pudding, chocolate, sponge cake, waffle, donut, cookie, biscuit, cake, cream, Japanese cracker, Japanese rice biscuit, etc., breads, rice cakes, buns with a bean-jam filling, uiro (a kind of rice cake made of rice powder and sugar), sweet bean jams, youkan (sweetened and jellied bean paste), mizuyoukan (soft sweetened and jellied bean paste), jelly, candy, pastas, macaroni, rice, premixed powders, artificial meat, canned products, fish food products such as kamaboko (boiled fish paste), chikuwa (a tubular role of boiled or grilled fish paste), tempura, etc., various seafood delicacies, fish boiled in soy sauce and fermented fish meat such as fermented fish products, dried mirin-seasoned fish, etc., and processed meat products such as ham, sausage, bacon, etc., soy sauce, miso (fermented soybean paste), processed seaweed, fish boiled in soy sauce, tangle roll, various drinks, various seasonings, daily dish such as potato salad, simmered meat and potatoes, chikuzen-ni (various vegetable and chicken simmered and flavored in soy sauce), boiled beans, pastes, boiled and smashed beans.

Amino acid includes, for example, aspartic acid, glutamic acid, glutamine, arginine, proline, methionine, histidine, phenylalanine, tryptophan, threonine, lysine, glycine, alanine, etc. Preferable amino acid may be branched-chain amino acids, for example, isoleucine, leucine, valine, etc. Amino acid may be used in the form of sodium salt etc.

In the present invention, it is preferable to add carnitine, acetylcarnitine, glutamic acid, amino acids (aspartic acid, cysteine, lysine, methionine, arginine, isoleucine, leucine), etc. into D-ribose. Further, vitamins or revitalizers may be added thereto. The vitamins to be added include, for example, Vitamin As, Vitamin Bs, Vitamin Cs, Vitamin Ds, Vitamin Es, nicotine acid, nicotinamide, pantothenic acid, panthenol, biotin, folic acid, etc. The revitalizers include, for example, pantethine, glucuronic acid, glucuronolactone, inositol, inositol hexanicotinate, ursodeoxycholic acid, orotic acid, γ-oryzanol, chondroitin sulfate, taurine, natural medicines having nutritional fortification activity, etc.

When D-ribose is used as an agent for improving depression-like symptoms, it is preferably used in the form of a drinkable preparation, and in addition thereto, it is used in the form of an oral liquid preparation such as syrup, etc., intravenous dosage forms such as injection, etc. or an oral solid preparation such as troches, lozenges, chewable tablets, etc. These dosage forms may be prepared in a conventional manner. The dosage of D-ribose may vary according to the administration routes, ages, body weights or conditions of patients to be treated, etc., but it is usually in the range of about 10 mg to about 100 g, preferably in the range of about 30 mg to about 5 g, more preferably in the range of about 100 mg to about 500 mg per day in adult. The dosage of D-ribose may be given once a day or more than once throughout the day. However, when a magnesium salt is simultaneously used, the dosage of D-ribose may be reduced to about ½ to about ⅕. In addition, D-ribose may be added to foods and drinks such as beverage or food in order to improve depression-like symptoms. For example, by adding to hospital diets, the willingness of hospitalized patients may be increased.

The dosage of a magnesium salt to be added to D-ribose may vary according to the administration routes, ages, body weights or conditions of the patients to be treated, but as a weight of magnesium, it is usually in the range of about 2 mg to about 500 mg, preferably in the range of about 5 mg to about 200 mg, more preferably in the range of about 10 mg to about 100 mg per day in adult. The dosage of magnesium salt may be given once a day or more than once throughout the day.

The dosage of an amino acid to be added to D-ribose may vary according to the administration routes, ages, body weights or conditions of the patients to be treated, but as a weight of amino acid, it is usually in the range of about 2 mg to about 500 mg, preferably in the range of about 5 mg to about 200 mg, more preferably in the range of about 10 mg to about 100 mg per day in adult. The dosage of amino acid may be given once a day or more than once throughout the day.

The dosage of carnitine to be added to D-ribose may vary according to the administration routes, ages, body weights or conditions of the patients to be treated, but as a weight of carnitine, it is usually in the range of about 2 mg to about 500 mg, preferably in the range of about 5 mg to about 200 mg, more preferably in the range of about 10 mg to about 100 mg per day in adult. The dosage of carnitine may be given once a day or more than once throughout the day.

The improving effect on the depression-like symptoms may be evaluated using an evaluation method for antidepressant. The evaluation method includes, for example, in addition to the method of observing the symptoms after administered to a human, methods using learned helplessness model, separated breeding model, forced swimming model, chronic stress-induced model, muricide (=mouse killing behavior) model (cf., Neuropsychopharmacology (in Japanese), 7 (6), p. 383-391 (1985)), reserpine-induced hypothermia competitive test (cf., Japan J. Pharmacol. 53, p. 451-461 (1990)), rotor rod test (J. Am. Pharm. Ass., 46, p. 208-209 (1957); Folia Phamacologica Japonica, 104, p. 39-49 (1994)), Tail Suspension test (cf., Courvoisier, S. et. al. In “Psychotropic drugs” ed. By Garattini, S. and Ghetti, V., p. 373, Elsevier, Amsterdam, 1957), Stress-related soaking fatigue model (cf., Igaku-no-Ayumi, 204 (5), p. 362-364 (2003)), etc.

### EXAMPLES

The present invention is illustrated by the following Examples, but the present invention should not be construed to be limited thereto.

### Example 1

Improving Effect of D-Ribose on Depression-Like Symptoms in Forced Swimming Test in Mice:

Test Method:

Male 10 ddY-strain mice (5 weeks old, purchased from Japan SLC, Inc.) were used for each group as test animals. The animals were housed in groups of 20 mice in a plastic-made cage (26×43×16 cm; Clea Japan Inc.), and they were kept in the animal room maintained at 23±2° C. with 30 to 80% humidity, and illuminated for 12 hr (6:30 to 18:30). The animals were allowed free access to pellet diets (CRF-1, Oriental Yeast, Co., Ltd.), and tap water.

The experiment was carried out on the four groups, such as the groups treated with D-ribose at doses of 30 mg/kg, 100 mg/kg, 300 mg/kg, and the control group. The animals were grouped based on the body weights which had been previously measured prior to the experiment so that the average body weight of each group becomes equal. D-ribose was dissolved in distilled water and administered orally at 10 ml/kg once a day and repeatedly for one week in mice. To the control group, distilled water was administered orally instead of aqueous D-ribose solution. The forced swimming test was a modification of the method of Porsolt et al. (cL, Nature, 166, p. 730-732 (1977)). Briefly, the animals were forced to swim twice, i.e., for 15 minutes prior to the treatment of a test compound or distilled water on the day before the final administration, and further for 5 minutes one hour after the final administration on the following day. That is, the mice were forced to swim in a clear polycarbonate-made measuring cylinder (internal diameter: 10 cm, height: 25 cm) containing water up to a height of 10 cm at a temperature of 250° C., and the duration of the immobility during the second swimming was recorded.

Statistical Processing:

The results were expressed as mean±standard error for each group. The significant differences between the groups were studied by Dunnett's multiple comparison test with a significance level of 5%.

Test Results:

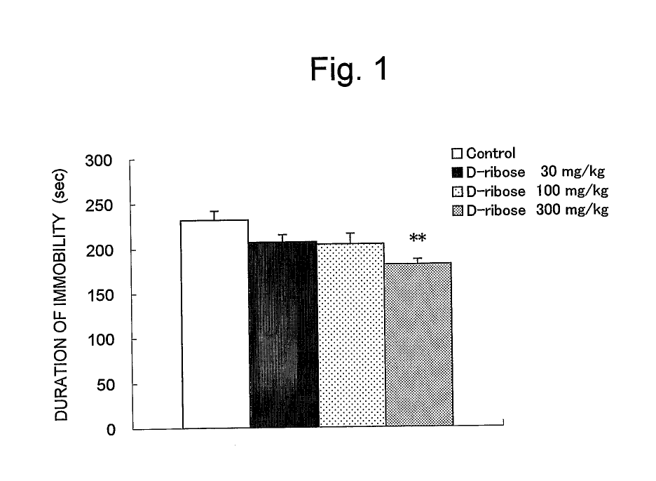

The results are shown in FIG. 1. In the group treated with 300 mg/kg, the duration of immobility was significantly reduced, and therefore, it was proved that D-ribose shows an improving activity of depression-like symptoms.

### Example 2

Improving Effect of a Combined Treatment of D-Ribose and Potassium Magnesium Aspartate on Depression-Like Symptoms in Forced Swimming Test in mice:

Method:

Male10 ddY-strain male mice (5 weeks old, purchased from Japan SLC, Inc.) were used for each group as test animals. The animals were kept under the same conditions as in Example 1. The experiment was carried out on the four groups, such as the groups treated with D-ribose 100 mg/kg, potassium magnesium aspartate (Mg.K aspartate) 50 mg/kg, and D-ribose 100 mg/kg+Mg.K aspartate 50 mg/kg, and the control group. The animals were grouped based on the body weights which had been previously measured prior to the experiment so that the average body weight of each group becomes equal. D-ribose and potassium magnesium aspartate were dissolved in distilled water and administered orally at 10 ml/kg once a day and repeatedly for one week. To the control group, distilled water was administered orally instead of aqueous solution of test compound.

Statistical Processing:

The forced swimming test, the measurement of the duration of the immobility during the swimming, and the expression of the results, and the test of significant differences between the groups were all carried out in the same manner as in Example 1.

Test Results:

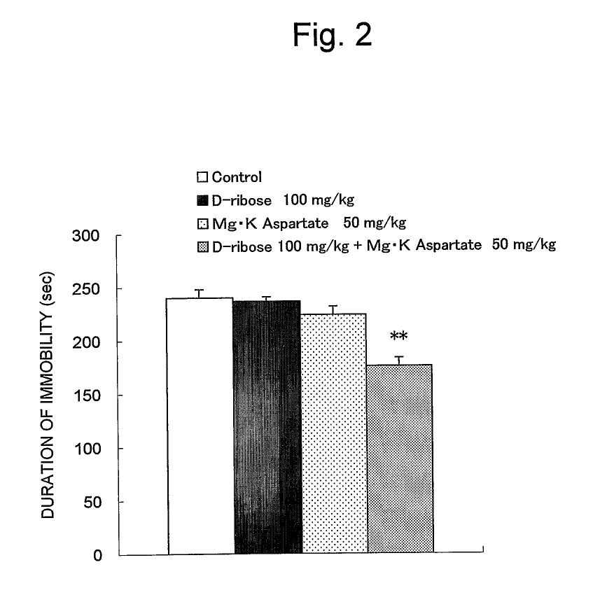

The results are shown in FIG. 2. When D-ribose 100 mg/kg or Mg.K aspartate 50 mg/kg was administered alone, the duration of immobility was not significantly reduced in the both groups, while when D-ribose 100 mg/kg and Mg.K aspartate 50 mg/kg were administered together, the duration of immobility was significantly reduced. Thus, it was proved that a magnesium salt such as Mg.K aspartate may apparently enhance the improving effect of D-ribose on the depression-like symptoms.

### Example 3

Improving Effect of D-Ribose on Depression-Like Symptoms in Reserpine-Induced Hypothermia Competitive Test:

Test Method:

Male 8 ddY-strain mice (5 weeks old, purchased from Japan SLC, Inc.) were used for each group as test animals. The animals were kept under the same conditions as in Example 1. The experiment was carried out on the four groups such as the groups treated with D-ribose at doses of 30 mg/kg, 100 mg/kg, 300 mg/kg, and the control group. The animals were grouped based on the body temperature (rectal temperature) which had been previously measured prior to the experiment so that the average body temperature of each group becomes equal. D-ribose was dissolved in distilled water and administered orally at 10 ml/kg once a day and repeatedly for one week. To the control group, distilled water was administered orally instead of aqueous D-ribose solution. The reserpine-induced hypothermia competitive test was a modification of the method of Wachtel (cf., Neuropharmacology, 22 (3), p. 267-272 (1983)). Namely, a solution of reserpine (Daiichi Pharmaceutical Co. Ltd.) in an aqueous propylene glycol solution was subcutaneously administered to the mice in each group at a dose of 1 mg/kg (i.e., in an amount of 10 ml/kg) just before administration of a test compound or distilled water, and subsequently, D-ribose or distilled water were administered orally to the mice, and then the rectal temperature of each mouse was measured on 1, 2, 4, 6 and 8 hours after administration of D-ribose or distilled water. In the experiment, the reserpine treatment and the administration of the test compound were carried out during from 10:00 AM to 11:00 AM.

Statistical Processing:

The results were expressed as mean±standard error in each group. The significant differences between the groups were studied by Bonferroni/Dun multiple comparison test with a significance level of 5%.

Test results:

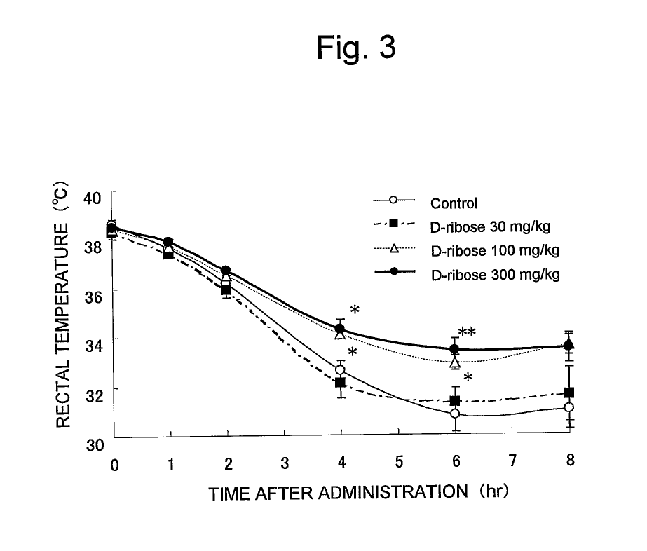

The results are shown in FIG. 3. In the group treated with D-ribose at a dose of 30 mg/kg, the reserpine-induced hypothermia was not significantly alleviated, while in the groups treated with D-ribose at doses of 100 and 300 mg/kg, the reserpine-induced hypothermia was significantly and nearly dose-relatively alleviated 4 or 6 hours after the administration of D-ribose, by which it was proved that D-ribose shows an improving effect on depression-like symptoms.

### Example 4

Improving Effect of a Combined Treatment of D-Ribose and Potassium Magnesium Aspartate on Depression-Like Symptoms in Reserpine-Induced Hypothermia Competitive Test:

Test method:

Male 8 ddY-strain mice (5 weeks old, purchased from Japan SLC, Inc.) were used for each group as test animals. The animals were kept under the same conditions as in Example 1. The experiment was carried out on the three groups such as the groups treated with D-ribose 100 mg/kg, D-ribose 100 mg/kg+Mg.K aspartate 50 mg/kg, and the control group. D-ribose and Mg.K aspartate were dissolved in distilled water and administered orally to the mice at 10 ml/kg. To the control group, distilled water was administered orally. The grouping, the administration of reserpine and the measurement of body temperature were carried out in the same manner as in Example 3.

Statistical Processing:

The results were expressed as mean±standard error for each group. The significant differences between the groups were studied by Bonferroni/Dunn multiple comparison test with a significance level of 5%.

Test Results:

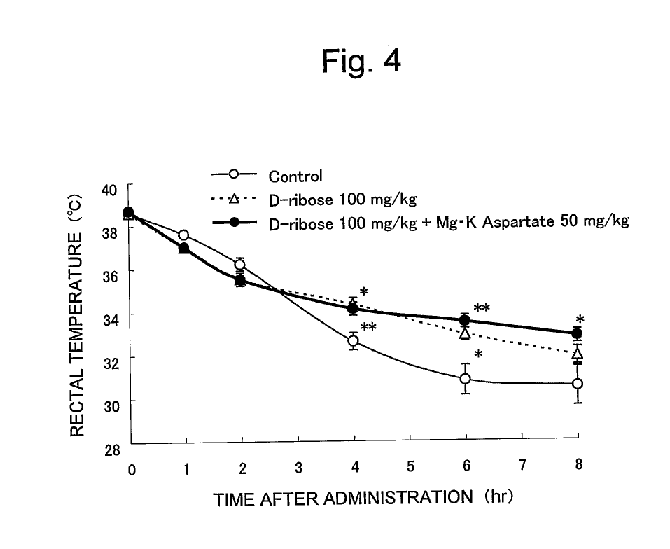

The results are shown in FIG. 4. In the group treated with D-ribose alone at a dose of 100 mg/kg, the reserpine-induced hypothermia was significantly alleviated at 4 and 6 hours after the D-ribose treatment. On the other hand, in the group treated with a combination of D-ribose 100 mg/kg and Mg.K aspartate 50 mg/kg, the reserpine-induced hypothermia was significantly alleviated at 4, 6 and 8 hours after the treatment. Thus, it was proved that a magnesium salt such as Mg.K aspartate may enhance and sustain the improving effect of D-ribose on depression-like symptoms.

### Example 5

Anti-Fatigue Effects in the Soaking Fatigue Test in Rats:

Test Method:

Male 10 SD-strain rats (7 weeks old, purchased from Japan SLC, Inc.) were used for each group as test animals. The animals were grouped based on the body weights which had been previously measured prior to the experiment so that the average body weight of each group becomes equal. The animals were kept under the same conditions as in Example 1. The experiment was carried out on the 3 groups, such as the groups treated with D-ribose at doses of 300 mg/kg, 1000 mg/kg, and the control group. To the D-ribose-treated groups, D-ribose was dissolved in distilled water for injection in a predetermined amount for each body weight, and the resulting aqueous solution was mandatorily administered orally to the rats at a dose of 10 ml/kg. To the control group, distilled water for injection was mandatorily administered orally instead of aqueous D-ribose solution at a dose of 10 ml/kg.

The soaking fatigue test in rats was carried out by a modification of the method of Tanaka (cf., Igaku-no-Ayumi, 204 (5), 362-364 (2003)). Briefly, the rats in each group were kept in a cage where water at 23±1° C. was filled at a water depth of about 1.5 cm for 5 days, during which D-ribose was orally administered to the rats twice a day at 10 A.M. and 3 P.M. with divided amount of the daily dose (300 mg/kg or 1000 mg/kg), i.e., at each time 150 mg/kg or 500 mg/kg, respectively, for 5 days in a row. Twenty-four hours after the last administration, the rats were loaded with a weight (about 20 g, about 8°% of the body weight) at a position of about ⅘ distant from the root. Each rat was forced to swim individually in a clear acrylic cylindrical water bath (inner diameter: 25 cm, height: 60 cm) containing warm water (25±2° C., water-depth: 50 cm). The time from the beginning of swimming until the nose of the rat sank down into the water for more than 10 seconds was measured.

Statistical Processing:

The results were expressed as mean±standard error for each group. The significant differences between the groups were studied by Bonferroni/Dun multiple comparison test with a significance level of 5%.

Test Results:

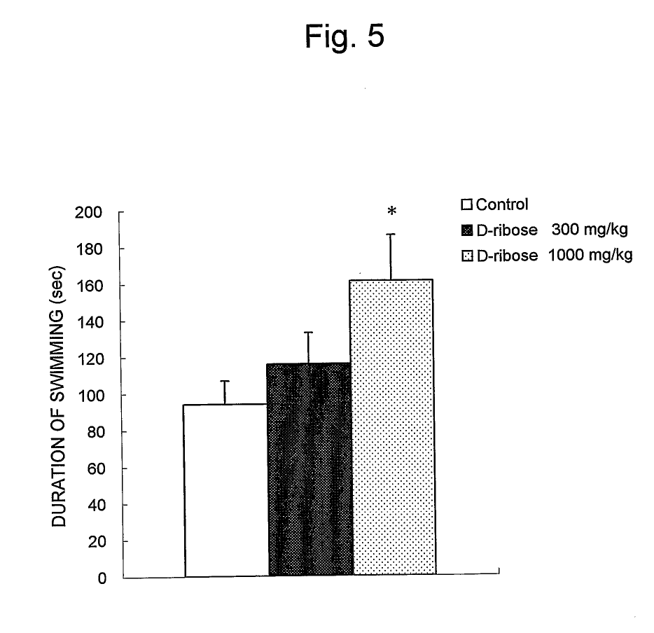

The results are shown in FIG. 5. In the group treated with D-ribose at a dose of 300 mg/kg, any significant anti-fatigue effect was observed, while the duration of swimming in the group treated with D-ribose at a dose of 1000 mg/kg was about 70% longer than that in the control group, which means that there is a significant anti-fatigue effect on the stress-related fatigue caused by breeding under soaking for 5 days. As is clear from the above, it was shown that D-ribose shows an improving effect on mental fatigue caused by stress, etc., and environmental fatigue caused by environmental factors such as noise, vibration or high-humidity.

### Example 6

Evaluation of Effect of Maintaining Concentration in Human:

The effect of maintaining concentration by D-ribose in human can be evaluated by the test using a random number table (cf., Sports Mental Training for Winning Games, written by Yoshihide TAKAHATA, published by NATSUME CO, LTD., 2003).

Twenty healthy volunteers are grouped into two groups, i.e., the D-ribose 2.2 g/day-treated group, and the control group. The volunteers in the D-ribose 2.2 g/day-treated group take an aqueous solution of D-ribose (2.2 g), a roasted green tea extra (0.24 g) and sucralose (2 mg) in distilled water for injection per day. The volunteers in the control group take an aqueous solution of dextrin (2.2 g), a roasted green tea extra (0.24 g) and sucralose (2 mg) in distilled water per day. In both groups, the volunteers take the daily amount of the above for 7 days prior to the test, and said daily amount is divided and taken three times per day, i.e., before breakfast, before lunch and before dinner. On the test day, the whole daily amount is taken 30 minutes prior to the test.

The evaluation test for concentration power is carried out as follows. First, random numbers between 00 to 99 are generated, and a table having 100 cells (10 columns×10 rows) to which a random number thus generated is allocated individually to each cell is prepared. Then, a volunteer is made to mark off cells in number order, i.e., starting from 00, 01, 02, likewise. Then, the number of marked cells is compared between both groups and the concentration power is evaluated therefrom.

Further, after one-month washout period from the above evaluation test for concentration power, the volunteers in both groups are exchanged each other, and then the same test is carried out again on the volunteers. Combining the results of these two tests, the effect of maintaining concentration by D-ribose is comprehensively evaluated.

As a result, it is found that the concentration power of human is dominantly maintained by the administration of D-ribose.

### Comparative Example 1

Comparison Test of Improving Effects of D-Ribose and Glucose on Depression-Like Symptoms in Forced Swimming Test in Mice:

Test Method:

Male 10 ddY-strain mice (5 weeks old, purchased from Japan SLC, Inc.) were used for each group as test animals. The animals were grouped based on the body weights which had been previously measured prior to the experiment so that the average body weight of each group becomes equal. The animals were kept under the same conditions as in Example 1. The experiment was carried out on the four groups, such as the groups treated with D-ribose at doses of 100 mg/kg and 300 mg/kg, treated with glucose at a dose of 300 mg/kg, and the control group. The corresponding amount of D-ribose per kg of body weight was dissolved in distilled water for injection, and administered orally in force at 10 ml/kg repeatedly for one week to the mice in two groups. A solution of glucose in a distilled water prepared likewise was administered orally in force at 10 ml/kg repeatedly for one week to the mice in one group. To the control group, distilled water for injection was administered orally instead of aqueous D-ribose solution at 10 ml/kg repeatedly for one week. The experiment was carried out by a modification of the method of Porsolt et al. (cf., Nature, 166, p. 730-732 (1977)).

Statistical Processing:

The results were expressed as mean±standard error for each group. In the groups treated with D-ribose at doses of 100 mg/kg and 300 mg/kg, dose-depending reduction in the duration of immobility was observed, by which it was confirmed that D-ribose shows an improving effect on depression-like symptoms. On the other hand, in the group treated with glucose at a dose of 300 mg/kg, the duration of immobility is not significantly reduced, by which it was confirmed that glucose shows no improving effect on depression-like symptoms.

### INDUSTRIAL APPLICABILITY

As mentioned above, the present invention provides an agent for improving depression-like symptoms comprising D-ribose. The agent for improving depression-like symptoms of the present invention may improve and alleviate various symptoms such as hypobulia, general fatigue, sluggishness, enervation, deterioration in concentration, memory impairment, abnormal sensation/obtundation such as impaired sight, decline in thinking power, indefinite complaint, drop in operation efficiency, or feeling of malaise, etc.

1. A method for improving depression-like symptoms, which comprises:
administering to a patient in need thereof an effective amount of D-ribose.

2. The method for improving depression-like symptoms according to claim 1, wherein the depression-like symptoms are hypobulia, general fatigue, sluggishness, enervation, deterioration in concentration, memory impairment, abnormal sensation/obtundation such as impaired sight, decline in thinking power, indefinite complaint, drop in operation efficiency, or feeling of malaise.

3. The method for improving depression-like symptoms according to claim 1 or 2, wherein the depression-like symptoms are depression-like symptoms accompanied by mental overstrain or mental disorder.

4. The method for improving depression-like symptoms according to claim 1 or 2, which comprises administering D-ribose in an amount of 10 mg to 100 g per day for an adult.

5. The method for improving depression-like symptoms according to claim 1 or 2, which further comprises administering at least one of a magnesium salt, an amino acid and carnitine.

6. The method for improving depression-like symptoms according to claim 1 or 2, which further comprises administering potassium magnesium aspartate.

7. A composition, which comprises:
D-ribose; and
at least one of a magnesium salt, an amino acid and carnitine.

8. A food or drink for improving depression-like symptoms, which comprises D-ribose.

9. A method for improving mental fatigue, which comprises:
administering to a subject in need thereof and effective amount of D-ribose.
